(19) United States
(12) Plant Patent Application Publication
(10) Pub. No.: US 2018/0279533 P1
(43) Pub. Date: Sep. 27, 2018

(54) Calibrachoa Plant Named 'KLECA16337'

(50) ;

(71) Applicant: Klemm+Sohn GmbH & Co. KG, Stuttgart (DE)

(72) Inventors: Nils Klemm, Stuttgart (DE); Anita Stöver, Ostfildern (DE)

(73) Assignee: Klemm+Sohn GmbH & Co. KG, Stuttgart (DE)

(21) Appl. No.: 15/731,867

(22) Filed: Aug. 16, 2017

(30) Foreign Application Priority Data
Mar. 27, 2017 (CA) 17/9144

(51) Int. Cl. A01H 6/82

(52) U.S. Cl. PLT/413

(57) ABSTRACT

A new Calibrachoa plant particularly distinguished by having yellow flowers with darker yellow centers and a semi-trailing and mounding habit, is disclosed.

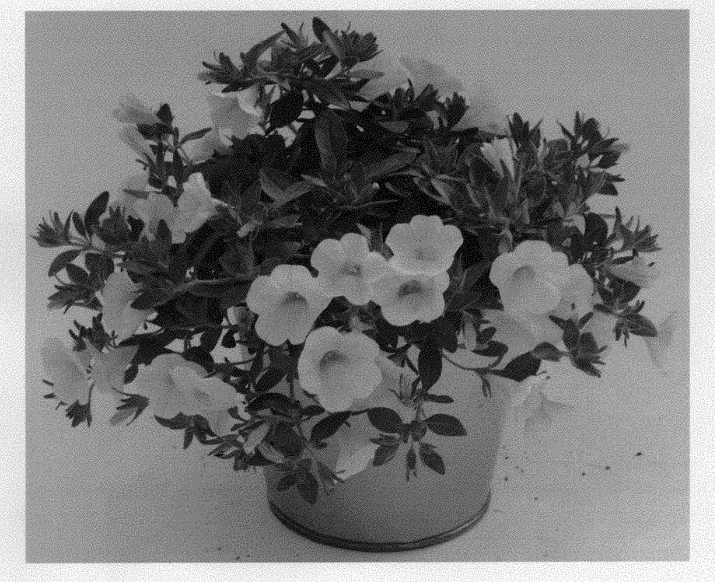

### CROSS-REFERENCE TO RELATED APPLICATIONS

This application claims priority to and the benefit of Canadian Plant Breeders Rights application filed on Mar. 27, 2017, Application No. 17/9144, the entire contents of which are incorporated herein by reference for all purposes.

Genus and species: Calibrachoa sp.

Variety denomination: ‘KLECA16337’.

### BACKGROUND OF THE NEW PLANT

The present invention comprises a new and distinct variety of Calibrachoa, botanically known as Calibrachoa sp., and hereinafter referred to by the variety name ‘KLECA16337’. ‘KLECA16337’ originated from a controlled-cross performed in summer 2012 between the proprietary female Calibrachoa parent ‘CA-2011-0508’ (unpatented), and the proprietary male Calibrachoa parent ‘CA-2012-0078’ (unpatented), in Stuttgart, Germany.

The seeds from the cross were sown and plants were grown in a greenhouse for evaluation, where an individual plant designated ‘KLECA16337’ was selected from the group of plants in May 2013 in Stuttgart, Germany. In May 2013, ‘KLECA16337’ was first vegetatively propagated by terminal tip cuttings. ‘KLECA16337’ was found to reproduce true to type in successive generations of asexual propagation via tissue culture and terminal tip cuttings.

### SUMMARY

The following are the most outstanding and distinguishing characteristics of this new variety when grown under normal horticultural practices in Stuttgart, Germany.
1. Yellow flowers with darker yellow centers; and
2. A semi-trailing and mounding habit.

### DESCRIPTION OF THE PHOTOGRAPH

This new Calibrachoa plant is illustrated by the accompanying photograph which shows the whole plant, including the habit, the foliage, the flowers, and the buds.

The photograph is of a 7-week-old plant grown in a pot outdoors in Stuttgart, Germany in April 2015. The colors shown are as true as can be reasonably obtained by conventional photographic procedures.

### DESCRIPTION OF THE NEW VARIETY

The following detailed descriptions set forth the distinctive characteristics of ‘KLECA16337’. The data which define these characteristics were collected from asexual reproductions carried out in Stuttgart, Germany. Data was collected on 20-week-old plants grown in an outdoor field in Stuttgart, Germany. Color references are to The R.H.S. Colour 5^{th} Edition Chart of The Royal Horticultural Society of London (R.H.S.) (2007).
Classification:
   Family.—Solanaceae.
   Botanical.—Calibrachoa sp.
   Common.—Calibrachoa.
   Designation.—‘KLECA16337’.
Parentage:
   Female parent.—Proprietary Calibrachoa ‘CA-2011-0508’ (unpatented).
   Male parent.—Proprietary Calibrachoa ‘CA-2012-0078’ (unpatented).
Plant:
   Life cycle.—Annual.
   Habit.—Semi-trailing and mounding.
   Height (from soil line to top of foliage).—10.0 cm.
   Spread.—45.0 cm.
   Propagation.—Vegetatively propagated by tip cuttings and tissue culture.
   Time to produce a rooted cutting.—3 weeks.
Stems:
   Stem color.—RHS 145A (Yellow-green).
   Pubescence.—Absent.
   Diameter.—0.2 cm to 0.3 cm.
   Length.—25.0 cm. From soil line to first node: 2.0 cm. Total length: 27.0 cm.
   Internode length.—1.5 cm to 3.0 cm.
Leaves:
   Arrangement.—Opposite.
   Shape.—Ovate.
   Apex.—Obtuse.
   Base.—Attenuate.
   Margin.—Ciliate.
   Surface texture.—Smooth.
   Surface pubescence.—Absent.
   Venation pattern.—Arcuate.
   Venation color.—Upper surface: RHS 144C (Yellow-green). Lower surface: RHS 144D (Yellow-green).
   Length.—2.0 cm.
   Width.—0.9 cm.
   Color.—Upper surface: RHS N137A (Green). Lower surface: RHS 137B (Green).
   Fragrance.—Absent.
Flowers:
   Flower type.—Determinate inflorescence with single flowers.
   Flowering requirements.—A minimum of light sun is required.
   Duration of flowers on the plant.—6 days.
   Shape.—Funnel-form.
   Fragrance.—Absent.
   Flower depth.—2.8 cm.
   Flower tube length.—1.8 cm.
   Flower tube diameter.—0.6 cm.
   Flower diameter.—3.2 cm.
   Pedicel color.—RHS 145A (Yellow-green).
   Pedicel length.—1.2 cm.
   Pedicel diameter.—0.1 cm.
Flower buds:
   Surface texture.—Smooth.
   Length.—1.9 cm.
   Diameter.—0.3 cm.
   Shape.—Irregular to oblong.
   Color.—RHS 4C (Yellow).
Peduncle:
   Length.—1.2 cm.
   Diameter.—0.1 cm.
   Color.—RHS 145A (Yellow-green).
   Surface texture.—Smooth.
   Anthocyanin color.—Absent.
Petals:
   Shape.—Obcordate.
   Length.—1.5 cm.
   Apex.—Rounded.
   Margin.—Entire, with weak undulation.
   Base.—Open and fused in the tube.
   Pubescence.—Absent.
   Lobe length.—1.5 cm.
   Lobe width.—1.6 cm.
   Color.—Lobe color: Upper surface: Main color is RHS 4D (Yellow) and is RHS 6C (Yellow) at the transitions to the corolla tube. Lower surface: RHS 4D (Yellow). Corolla tube color: Inner surface: RHS 9B (Yellow). Outer surface: RHS 9D (Yellow).
   Fragrance.—Absent.
Sepals:
   Calyx.—Funnel-shaped.
   Shape.—Lanceolate.
   Apex.—Acute.
   Margin.—Entire.
   Length.—1.3 cm.
   Diameter.—0.3 cm.
   Color.—Upper surface: RHS 143B (Green). Lower surface: RHS 143C (Green).
Reproductive organs:
   Stamen.—Quantity per flower: 5. Anther color: RHS 7A (Yellow). Filament color: RHS 145C (Yellow-green). Filament length: 0.8 cm. Pollen color: RHS 7A (Yellow). Pollen amount: Medium.
   Pistil.—Ovary, placenta arrangement: Ovate. Pistil number per flower: 1. Pistil length: 1.1 cm. Stigma color: RHS 143B (Green). Style length: 0.8 cm. Style color: RHS 150C (Yellow-green).
Fruit and seed set: Not observed.

### COMPARISON WITH PARENTAL LINES AND KNOWN VARIETY

‘KLECA16337’ is distinguished from the female and male parental lines as described in Table 1.

TABLE 1
Comparison with Parental Lines

| | | Female parent | Male Parent |
|---|---|---|---|
| Characteristic | ‘KLECA16337’ | ‘CA-2011-0508’ | ‘CA-2012-0078’ |
| Main flower color | Yellow | Orange-red | Pink |

‘KLECA16337’ is most similar to the commercial Calibrachoa plant named ‘KLECA15327’ (U.S. Plant Pat. No. 28,390), also known as MINIFAMOUS NEO ‘Deep Yellow’. Differences between the two varieties are described in Table 2.

TABLE 2
Comparison with Similar Variety

| Characteristic | ‘KLECA16337’ | ‘KLECA15327’ |
|---|---|---|
| Main flower color | RHS 4D | RHS 6C |
| Main color at transition to | RHS 6C | RHS 12A |
| corolla tube | | |
| Size of area of main color at | Medium | Large to very |
| transition to corolla tube | | large |

We claim:

1. A new and distinct variety of Calibrachoa plant named ‘KLECA16337’ as illustrated and described herein.
